(19) United States
(12) Patent Application Publication
(10) Pub. No.: US 2020/0060940 A1
(43) Pub. Date: Feb. 27, 2020

(54) BREASTFEEDING DEVICE

(71) Applicant: Children's Medical Center Corporation, Boston, MA (US)

(72) Inventors: Janet A. Conneely, Canton, MA (US); Olivia B. Oppel, Medway, MA (US); Salim Afshar, Cambridge, MA (US)

(21) Appl. No.: 16/500,436

(22) PCT Filed: Apr. 3, 2018
(86) PCT No.: PCT/US18/25816

§ 371 Date: Oct. 3, 2019

Related U.S. Application Data
(60) Provisional application No. 62/481,147, filed on Apr. 4, 2017.

(51) Int. Cl. A61J 9/06; A61F 2/52; A61J 9/00; A61J 11/00; A61J 11/04; A41C 3/04

(52) U.S. Cl. CPC A61J 9/0607; A61F 2/52; A61J 9/005; A61F 2002/526; A61J 11/0015; A61J 11/045; A41C 3/04; A61J 11/0005

(57) ABSTRACT

A breastfeeding device adapted to fit over a natural breast is provided. The device includes a reservoir that stores a received liquid and has an inner surface and an outer surface. Additionally, the device includes an access opening that is formed in the outer surface of the reservoir and is adapted to receive an attachment. The inner surface of the reservoir is attached onto a surface of the natural breast emulating a contour thereof.

### CROSS-REFERENCE TO RELATED APPLICATIONS

The present application claims the benefit under 35 U.S.C. § 119 of U.S. Provisional Patent Application No. 62/481,147 filed Apr. 4, 2017, the entire contents of which are hereby incorporated by reference herein.

### BACKGROUND

### 1. Technical Field

The present disclosure relates generally to a breastfeeding device, and more particularly, to a breastfeeding device which stores received liquid in a reservoir and is worn over a natural breast to facilitate natural breastfeeding.

### 2. Description of the Related Art

Recently, research has been conducted that shows the benefits of breastfeeding. For example, breastfeeding has been shown to improve the bond between a caregiver and an infant as well as provide significant health benefits for the infant. For example, breast milk has been proven to be one of the most valuable contributors to infant health and breastfeeding has been shown to reduce the risk of ear and respiratory infections, asthma, sudden infant death syndrome, diabetes, and obesity.

However, many caregivers suffer from the inability to provide an infant with breast milk through natural breastfeeding. For example, infants with anatomical challenges have difficulty in suckling at a natural breast, that is, they are unable to sufficiently latch onto a natural nipple. Other challenges leading to such suckling difficulties include tongue-tied infants in which the infant has limited mobility of the tongue, a parent has sore, cracked, or inverted nipples or has low milk production.

Accordingly, various devices and techniques for overcoming the above-described obstacles in breastfeeding have been developed. Some examples include nipple shields, breast shells, breast pumps, elongated nipple feeding systems, and the like. However, a number of these devices merely protect the nipple itself and do not provide improvements for infants struggling to suckle. The devices are also not capable of storing liquid or being worn by a caregiver to maintain or promote a human to human contact which has been shown to improve a bond between a parent and an infant.

Another developed conventional design is a bib like device which is adapted to receive a container therein which communicates with an opening in the bib. In other words, a nipple on the container that is inserted into the bib protrudes out from the fabric of the bib. Yet another developed design includes a device that is bell shaped and placed over the human breast. However, both of these devices require securement by straps attached to the device and then receive a nursing bottle there within. Neither one of these devices is capable of acting as a reservoir or being worn directly over a natural breast without requiring a strapping attachment.

Accordingly, the object of this disclosure is to provide a breastfeeding device that will more closely stimulate natural breastfeeding since common solutions including baby bottles, nipples, and other nursing devices do not resolve the physical limitations of caregivers wishing to experience the bonding benefit as well as the health benefits brought by breastfeeding.

### SUMMARY

The present disclosure provides a breastfeeding device that is adapted to fit over a natural breast to facilitate a more natural breastfeeding between a caregiver and infant.

According to one aspect, a breastfeeding device adapted to fit over a natural breast may include a reservoir which stores a received liquid and has an inner surface and an outer surface. The liquid may be received into the reservoir by a manual supply process or by a pump. The reservoir may be made of a flexible material. Additionally, an access opening is formed in the outer surface of the reservoir and is adapted to receive an attachment. The inner surface of the reservoir is attached onto a surface of the natural breast emulating a contour thereof.

In one exemplary embodiment, the attachment may be a nipple that protrudes from the outer surface of the reservoir for discharging the liquid from the reservoir. The nipple may be attached to the reservoir by a threaded connection. In particular, a threaded cap that holds the nipple may be attached to a threaded portion of the access opening to secure the nipple to the reservoir. The nipple provides an adjustable flow rate of the liquid discharged from the reservoir based on varied incisions in the nipple.

In another exemplary embodiment, the attachment may be a storage cap that seals the access opening of the reservoir. The attachment may also be a breast pump that supplies liquid into the reservoir. Further, the inner surface includes an adhesive that is releasably engaged with the natural breast to secure the breastfeeding device over the natural breast. Alternatively, the breastfeeding device may be worn inside a nursing brasserie.

In yet another exemplary embodiment, the reservoir is a sterile collection pouch and is made of a silicone material. The shape and size of the flexible reservoir may be customized based on the shape and size of the natural breast. The flexible reservoir may further include at least one sensor that measures liquid temperature, volume, suction amount, and oxidation. Additionally, the nipple itself receives and stores a portion of the liquid that is stored in the flexible reservoir to minimize the compression required for liquid discharge.

According to another aspect, a breastfeeding device is provided to fit onto a natural nipple of a natural breast. The breastfeeding device may include a flexible pouch having an inner surface and an outer surface, wherein a portion of the inner surface of the pouch is adapted to receive the natural nipple. Additionally, the breastfeeding device includes a receiving aperture that is formed in the inner surface of the pouch to be in liquid communication with the natural nipple. A nipple is attached to the outer surface of the flexible pouch and is adapted to receive liquid through the receiving aperture. The inner surface of the pouch emulates a contour of the natural breast and the natural nipple.

In one exemplary embodiment, the nipple stores a portion of the liquid received through the receiving aperture to minimize the compression required for liquid discharge. The pouch may further include an access opening in communication with a liquid storage device or a breast pump. The inner surface of the pouch further includes an adhesive releasably engaged with the natural breast to secure the breastfeeding device onto the natural breast. In another exemplary embodiment, the flexible pouch may be sealable. In yet another exemplary embodiment, the nipple may be formed integrally with the outer surface of the flexible pouch.

According to yet another aspect, a method of attaching a breastfeeding device is provided. The method may include filling a liquid into a reservoir having an inner surface and an outer surface and attaching a nipple attachment to an access opening formed in the outer surface of the reservoir. The inner surface of the reservoir is then disposed over the natural breast to secure the breastfeeding device and the inner surface emulates a contour of the natural breast.

In one exemplary embodiment, the disposing of the inner surface over the natural breast includes removing a protective cover from the inner surface to expose an adhesive releasably engaged with the natural breast. The method may further include removing the breastfeeding device from the natural breast and releasing the nipple attachment from the access opening. A cap may then be attached to the access opening to seal the access opening for storage of the liquid.

According to another aspect, a method of attaching a breastfeeding device onto a natural nipple of a natural breast is provided. The method may include fitting an inner surface of a pouch onto the natural nipple. The natural nipple is in liquid communication with a receiving aperture formed in the inner surface of the pouch. Additionally, a nipple is attached to an access opening formed in an outer surface of the pouch. The inner surface also emulates a contour of the natural nipple and the natural breast. In one exemplary embodiment, a liquid storage device or a breast pump may also be attached at an access opening formed in the outer surface of the pouch.

Notably, the present disclosure is not limited to the combination of the device elements as listed above and may be assembled in any combination of the elements as described herein.

Other aspects of the disclosure are disclosed infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1A illustrates a perspective view of the breastfeeding device according to an exemplary embodiment of the present disclosure;

FIG. 1B illustrates a rear perspective view of the breastfeeding device according to an exemplary embodiment of the present disclosure;

FIG. 1C illustrates a front view of the breastfeeding device according to an exemplary embodiment of the present disclosure;

FIGS. 2A-2B illustrate side views of the breastfeeding device according to an exemplary embodiment of the present disclosure;

FIG. 3 illustrates a rear view of the breastfeeding device according to an exemplary embodiment of the present disclosure;

FIGS. 4A-4B illustrate exemplary nipple attachments for the breastfeeding device according to an exemplary embodiment of the present disclosure;

FIGS. 5A-5C illustrate the attachment of the breastfeeding device over a natural breast according to an exemplary embodiment of the present disclosure;

FIGS. 6A-6B illustrate liquid storage within the reservoir and nipple of the breastfeeding device according to an exemplary embodiment of the present disclosure; and

FIG. 7 illustrates a breastfeeding device according to another exemplary embodiment of the present disclosure.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

### DETAILED DESCRIPTION

The presently disclosed subject matter will be described more fully hereinafter with reference to the accompanying drawings, in which some, but not all embodiments of the disclosure are shown. The presently disclosed subject matter may be embodied in many different forms and should not be construed as limited to the embodiments set forth herein; rather these exemplary embodiments are provided so that this disclosure will satisfy applicable legal requirements. Indeed, many modifications and other exemplary embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not limited to the specific embodiments disclosed and that modifications and other exemplary embodiments are intended to be included within the scope of the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

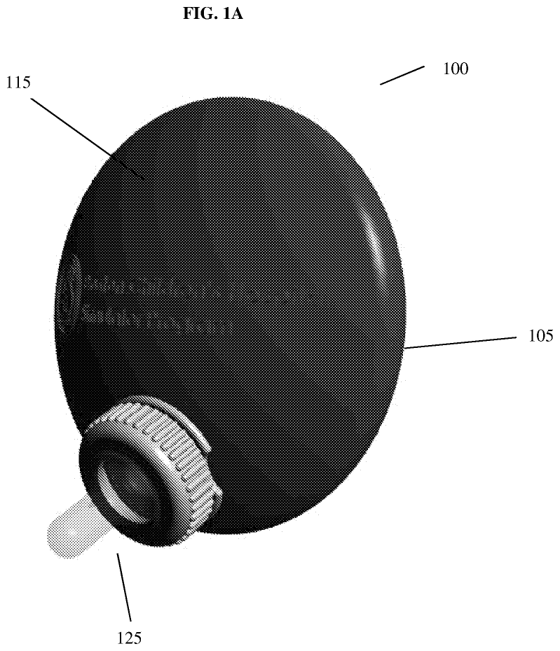

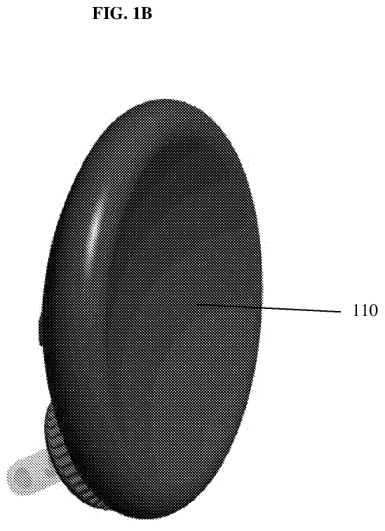

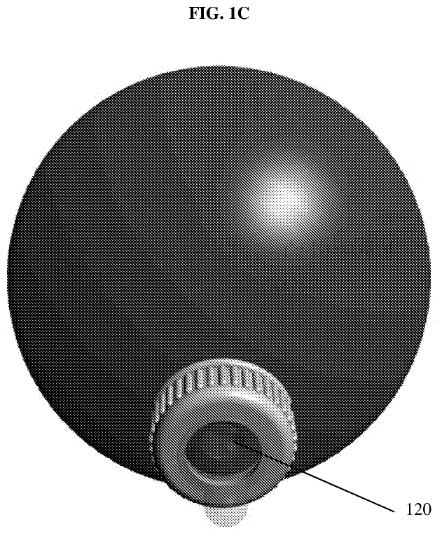

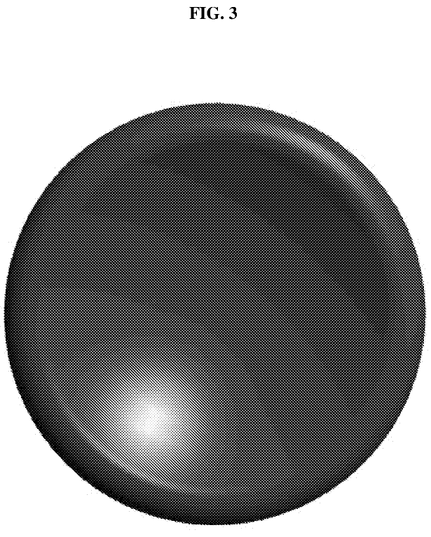

In one aspect, the present disclosure provides a breastfeeding device which stores received liquid in a reservoir and is worn over a natural breast to facilitate natural breastfeeding. FIGS. 1A-1C, 2A-2B, and 3 illustrate the breastfeeding device according to an exemplary embodiment of the present disclosure.

Referring to FIGS. 1A-1C, the breastfeeding device 100 according to an exemplary embodiment of the present disclosure may include a reservoir 105 that stores a liquid therein and has an inner surface 110 and an outer surface 115. The reservoir may be formed to be flexible so as to be able to emulate a contour of a natural breast. For example, the reservoir may be made of a silicone material, synthetic or non-synthetic material, organic silk, latex, pliable materials, and the like. That is, the reservoir is not limited to a particular material as long as it is flexible enough to emulate the contour of a natural breast while remaining a human safe material (e.g., safe to be worn on the skin for an extended period of time). This flexible material thus provides a caregiver with a more natural aesthetic. The reservoir may also be formed in any shape and size. For example, the size of the reservoir may correspond to standard brasserie sizes. Alternatively, the reservoir may be pre-molded into a personalized shape and size based on the caregiver.

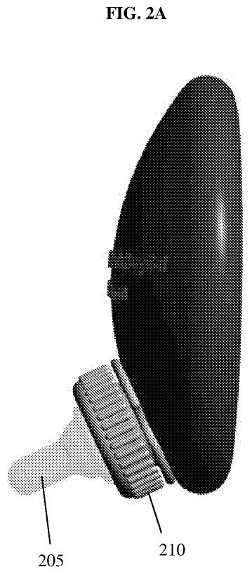

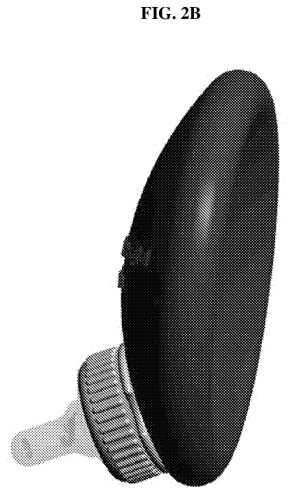

Further, the breastfeeding device 100 may include an access opening 120 that is formed in the outer surface 115 of the reservoir 105. The access opening 120 is adapted to receive an attachment 125. For example, as shown in FIG. 2A, the attachment 125 may be a nipple 205 onto which an infant may latch to engage with the breastfeeding device. The nipple 205 may be a commercially available nipple. The nipple 205 specifically protrudes out from the outer surface 115 of the reservoir 105 for discharging a liquid from within the reservoir 105. The nipple 205 may be attached to the reservoir 105 by a threaded connection. Particularly, a threaded cap 210 that holds the nipple 205 may be attached to a threaded portion of the access opening 120 to secure the nipple 205 to the reservoir 105. However, the exemplary embodiment is not limited thereto and the nipple may be secured to the reservoir using other well-known attachment means or by using a commercially available cap.

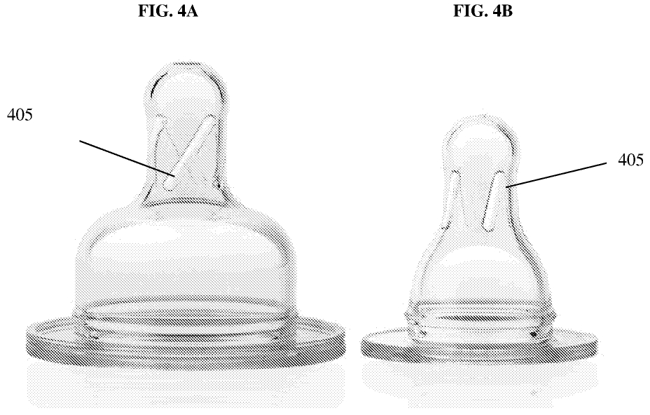

The nipple 205 advantageously provides an adjustable flow rate of the liquid discharged from the reservoir 105. As shown in FIGS. 4A-4B, the adjustable flow rate is achieved by incisions 405 formed in the nipple 205. That is, the incisions 405 are varied to thus adjust the flow rate. Accordingly, a caregiver is capable of attaching a nipple with an appropriate flow rate for different infants. For example, an infant of 2 months may require a different flow rate than an infant of 10 months. Thus, the breastfeeding device is adaptable to infants of all ages.

According to another exemplary embodiment, the attachment 125 may be a storage cap that is used to seal the access opening 120 of the reservoir 105. The storage cap allows the breastfeeding device 100 to be completely sealed from leakage. The breastfeeding device is thus both removable and portable while storing liquid. For example, the storage cap allows for the device to easily be placed in a refrigerator for later use or can be pre-filled with a liquid and is then portable for a later use. Thus, a caregiver can avoid the need of having to fill the reservoir when in an inconvenient location. Multiple devices can also be filled to have ready for use when needed. Thus, user convenience is greatly improved by having a device that is adapted to both be a storage compartment and a dispensing device while being portable.

According to yet another exemplary embodiment, the attachment 125 may be a breastfeeding pump. For example, the access opening 120 may receive a breast pump connector in communication with the breast pump. Thus, a user can readily fill the reservoir 105 of the device 100 using a breast pump and then seal the reservoir with a cap for later use or attach the nipple 205 for immediate use. That is, the breastfeeding device 100 according to the exemplary embodiment of the present disclosure may be filled either by the use of a breastfeeding pump (or another type of mechanical pump system) or by a manual process. The breastfeeding device, however, is not limited to these filing processes and may be adapted to be filled by any known process.

Furthermore, the flexible reservoir 105 is a sterile collection pouch that may easily be cleaned for reuse. The reservoir 105 may be made of various materials that are both flexible and safe for skin contact. For example, the reservoir 105 may be made of a silicon material, an organic silk, a pliable plastic, or other well-known materials. In some exemplary embodiments, various sensors may be disposed within the reservoir. For example, a temperature sensor may be included within the reservoir to measure the temperature of the liquid and a volume sensor may be included therein to determine an amount of liquid available within the reservoir. Additionally, a sensor may be provided to measure the amount of suction required to discharge the liquid through the nipple or an amount of oxidation may be measured. Yet another example includes a sensor capable of measuring the suction of an infant on the nipple. In particular, the suction attempt of an infant may be determined by detecting the pressure and displacement of the nipple. This measurement is useful for children with low muscle tone in which the muscle strength is strengthened over time. However, the sensors are not limited to these specifically discussed sensors and various other sensors may also be mounted within the reservoir or on the breastfeeding device itself.

These sensors may be integrated into one single sensor. Further, the data gathered by these sensors may be transmitted to a user device. For example, a user may receive information regarding a remaining amount of liquid within the reservoir. Alternatively, the reservoir may include a blocking means which is enabled when, for example, the temperature of the liquid within the reservoir greater than a safe temperature (e.g., a safe temperature for the infant to receive the liquid). In other words, the hot liquid may be prevented from being discharged through the nipple, thus preventing potential harm and injury to the infant.

Moreover, when the flexible reservoir 105 has been filled with liquid by a manual process, a breast pump, or another filling process, the nipple 205 attachment may be attached to the access opening 120. Alternatively, the nipple 205 may be attached to the access opening 120 after the breastfeeding device 100 has been attached for use. That is, after the breastfeeding device 100 has been either attached over the natural breast or placed within a nursing brasserie. The attachment of the breastfeeding device 100 over the natural breast will be described in more detail below.

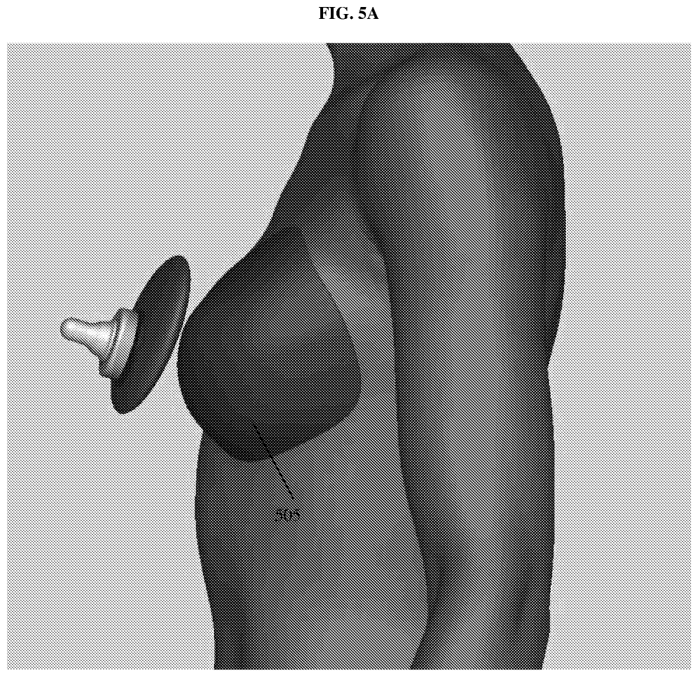

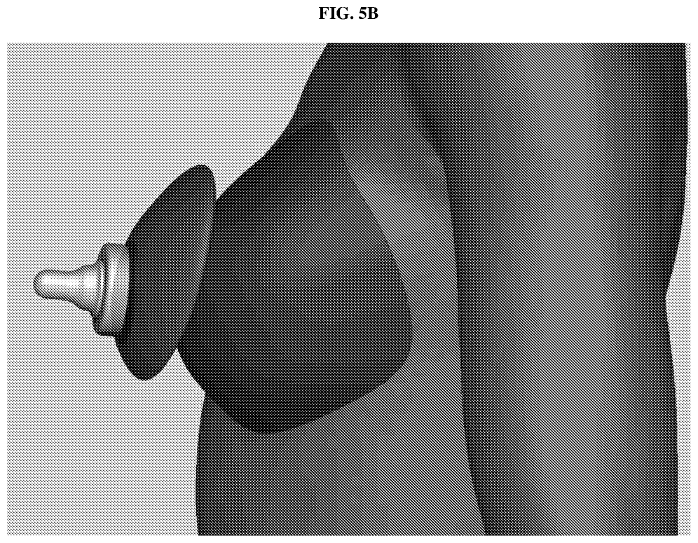

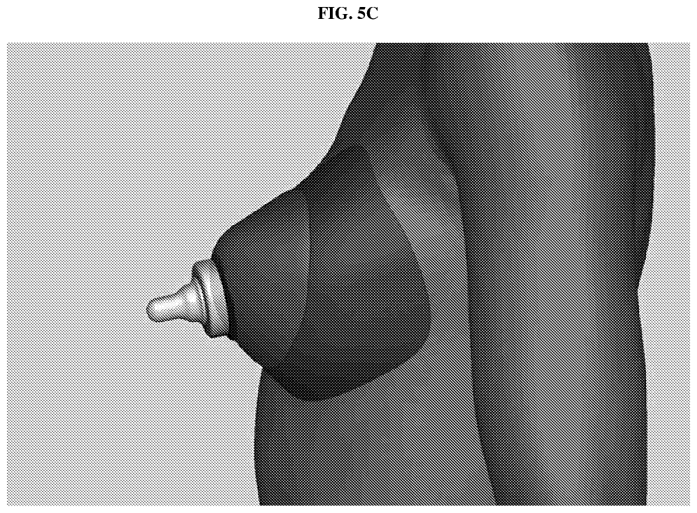

When the flexible reservoir 105 is filled with liquid and the nipple 205 has been attached thereto, the breastfeeding device 100 may be fit over a natural breast of a user or caregiver. In particular, the inner surface 110 of the reservoir 105 may be attached onto a surface 505 of the natural breast, as shown in FIGS. 5A-5B. As the reservoir contacts the surface 505 of the natural breast, due to the flexible material, the reservoir 105 emulates a contour of the natural breast. Particularly, FIG. 5C shows how the flexible reservoir 105 changes shape to correspond to the shape of the user's natural breast. This shape change provides a natural feeling to a user. That is, the reservoir most accurately fits over the breast to minimize discomfort for a user wearing the device. To avoid any additional attachments to secure the breastfeeding device to the user, the inner surface 110 of the reservoir 105 may include an adhesive. Accordingly, the breastfeeding device may be releasably engaged with the natural breast. The adhesive is a composition that allows for the surface to be reengaged with the surface of the natural breast over multiple uses. That is, the adhesive is not permanent and also continues to exhibit adhesive characteristics over many uses. A protective cover may also be further provided over the adhesive to protect the adhesive layer for next uses. That is, prior to attaching the breastfeeding device onto the natural breast, the protective cover may be removed from the inner surface of the reservoir to expose the adhesive. After use of the breastfeeding device, the protective cover may be replaced over the adhesive to protect the adhesive from dust or other particles.

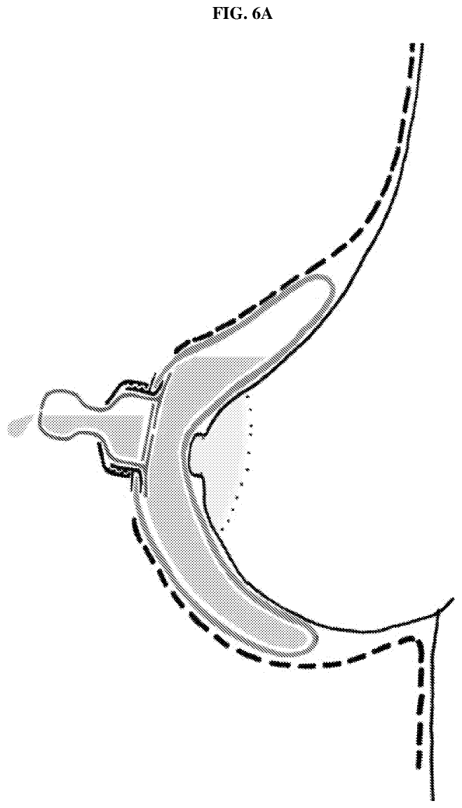

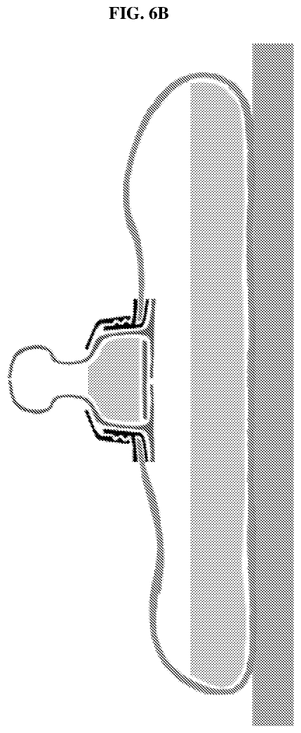

Referring to FIGS. 6A-6B illustrate, once the breastfeeding device has been fitted over the natural breast, the liquid may be stored within the reservoir. Further, due to the structural shape and configuration of the nipple, the nipple itself is also capable of holding/storing a particular amount of liquid received from the reservoir. The storage of the liquid within a portion of the nipple allows for minimal compression to be required to discharge the liquid through the nipple opening. Thus, an infant having difficulty to suckle is capable of more easily receiving liquid from the reservoir.

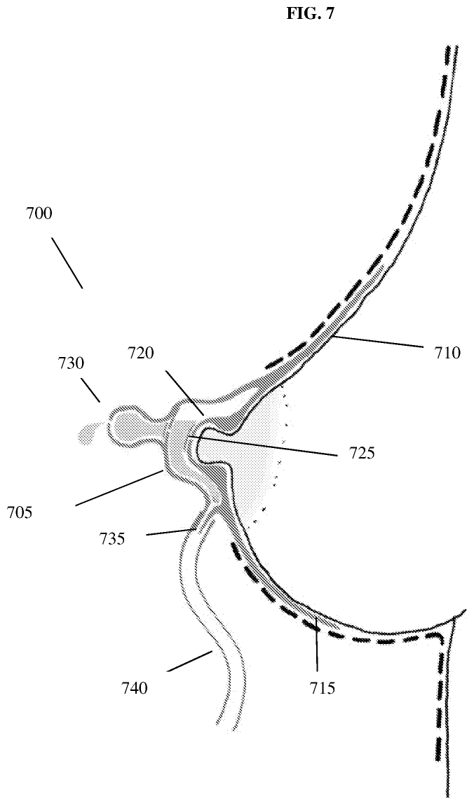

Accordingly to another exemplary embodiment of the present disclosure, the breastfeeding device may be adapted to fit onto a natural nipple of a natural breast. Particularly, as shown in FIG. 7, the breastfeeding device 700 may include a flexible pouch 705 that has an inner surface 710 and an outer surface 715. A portion 720 of the inner surface 710 of the pouch 705 is adapted to receive the natural nipple. That is, the portion 720 of the inner surface 710 may emulate a contour of the natural nipple. The inner surface 710 may also include a receiving aperture 725 to be in liquid communication with the natural nipple. Further, a nipple 730 may then be attached to the outer surface 715 of the pouch 705. The nipple 730 is adapted to receive liquid through the receiving aperture 725. The inner surface 710 of the pouch 705 also emulates a contour of the natural breast. In other words, the inner surface 710 is capable of changing shape to correspond to the entire breast, including the nipple. The portion of the surface that is attached only to the breast, instead of the nipple, may include an adhesive to be releasably secured to the user. Alternatively, the entire inner surface of the pouch may include the adhesive to provide a further seal between the user and the breastfeeding device.

In some exemplary embodiments, the pouch 705 may further include an access opening 735 in communication with a liquid storage device or a breast pump. For example, the access opening 735 may be configured to receive a communication line 740 that provides liquid communication between the pouch 705 and the storage device or breast pump (not shown). Accordingly, the breastfeeding device may alternatively be used in conjunction with a breast pump or similar device. This advantageously protects a natural nipple that may be sore or cracked from further susceptibility to injury by allowing the infant to suckle the nipple portion of the breastfeeding device. Also, if a caregiver experience difficulty with milk production, the breastfeeding device 700 of the present disclosure can be used in conjunction with both the receiving aperture 725 for when milk is capable of being produced and the communication line 740 to allow stored liquid to be administered to the infant. Accordingly, the breastfeeding device provides further adaptability based on user needs. The flexible pouch 705 may also be sealable to be capable of storing the liquid remaining therein. The nipple itself may also be formed integrally with the breastfeeding device thus providing a seamless device.

The breastfeeding device of the present disclosure provides numerous advantages. For instance, as discussed herein, the breastfeeding device is capable of providing a reservoir that is directly worn over a natural breast and reusable thus promoting a bond between an infant and caregiver. Importantly, the breastfeeding device facilitates breastfeeding by minimizing a compression required to discharge liquid from the reservoir, and thus, an infant can easily receive liquid therefrom. The reservoir pouch of the breastfeeding device is flexible such that it emulates a contour of a natural breast. Additionally, the breastfeeding device is attached to the caregiver by an adhesive and thus can be easily worn without requiring complex securing attachments.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

1. A breastfeeding device adapted to fit over a natural breast, comprising:
a reservoir that stores a received liquid and has an inner surface and an outer surface; and
an access opening formed in the outer surface of the reservoir and adapted to receive an attachment,
wherein the inner surface of the reservoir is attached onto a surface of the natural breast emulating a contour thereof.

2. The breastfeeding device of claim 1, wherein the reservoir is made of a flexible material.

3. The breastfeeding device of claim 1, wherein the reservoir receives liquid there into by a manual supply process or by a pump.

4. The breastfeeding device of claim 3, wherein the attachment is a nipple protruding from the outer surface of the reservoir for discharging the liquid from the reservoir.

5. The breastfeeding device of claim 4, wherein the nipple is attached to the reservoir by a threaded connection.

6. The breastfeeding device of claim 5, wherein a threaded cap that holds the nipple is attached to a threaded portion of the access opening to secure the nipple to the reservoir.

7. The breastfeeding device of claim 4, wherein the nipple provides an adjustable flow rate of the liquid discharged from the reservoir based on varied incisions in the nipple.

8. The breastfeeding device of claim 1, wherein the attachment is a storage cap that seals the access opening of the reservoir.

9. The breastfeeding device of claim 1, wherein the attachment is a breast pump that supplies the liquid into the reservoir.

10. The breastfeeding device of claim 1, wherein the inner surface includes an adhesive releasably engaged with the natural breast to secure the breastfeeding device over the natural breast.

11. The breastfeeding device of claim 1, wherein the breastfeeding device is worn inside a nursing brasserie.

12. The breastfeeding device of claim 2, wherein the flexible reservoir is a sterile collection pouch and is made of a silicone material.

13. The breastfeeding device of claim 1, wherein a shape and a size of the reservoir is customizable based on a shape and a size of the natural breast.

14. The breastfeeding device of claim 1, wherein the reservoir includes at least one sensor to measure liquid temperature, volume, suction amount, and oxidation.

15. The breastfeeding device of claim 4, wherein the nipple receives and stores a portion of the liquid stored in the reservoir to minimize the compression required for liquid discharge.

16. A breastfeeding device adapted to fit onto a natural nipple of a natural breast, comprising:
a flexible pouch having an inner surface and an outer surface, wherein a portion of the inner surface of the pouch is adapted to receive the natural nipple;
a receiving aperture formed in the inner surface of the pouch to be in liquid communication with the natural nipple; and
a nipple attached to the outer surface of the flexible pouch and adapted to receive liquid through the receiving aperture,
wherein the inner surface of the flexible pouch emulates a contour of the natural breast and the natural nipple.

17. The breastfeeding device of claim 16, wherein the nipple stores a portion of the liquid received through the receiving aperture to minimize the compression required for liquid discharge.

18. The breastfeeding device of claim 17, wherein the flexible pouch further includes an access opening in communication with a liquid storage device or a breast pump.

19. The breastfeeding device of claim 16, wherein the inner surface of the flexible pouch includes an adhesive releasably engaged with the natural breast to secure the breastfeeding device onto the natural breast.

20. The breastfeeding device of claim 16, wherein the flexible pouch is sealable.

21. The breastfeeding device of claim 16, wherein the nipple is formed integrally with the outer surface of the flexible pouch.

22. A method of attaching a breastfeeding device of claim 1 over a natural breast, comprising:
filling a liquid into a reservoir having an inner surface and an outer surface;
attaching a nipple attachment to an access opening formed in the outer surface of the reservoir; and
disposing the inner surface of the reservoir over the natural breast to secure the breastfeeding device,
wherein the inner surface emulates a contour of the natural breast.

23. The method of claim 22, wherein in disposing the inner surface over the natural breast, a protective cover is removed from the inner surface of the reservoir to expose an adhesive releasably engaged with a surface of the natural breast.

24. The method of claim 23, further comprising:
removing the breastfeeding device from the natural breast;
releasing the nipple attachment from the access opening; and
attaching a cap to the access opening to seal the access opening for storage of the liquid.

25. The method of claim 24, further comprising:
replacing the protective cover onto the inner surface of the reservoir.

26. A method of attaching a breastfeeding device of claim 16 onto a natural nipple of a natural breast, comprising:
fitting an inner surface of a pouch onto the natural nipple, wherein the natural nipple is in liquid communication with a receiving aperture formed in the inner surface of the pouch; and
attaching a nipple to an access opening formed in an outer surface of the pouch,
wherein the inner surface emulates a contour of the natural nipple and the natural breast.

27. The method of claim 26, further comprising:
attaching a liquid storage device or a breast pump at an access opening formed in the outer surface of the pouch.
